(19) United States
(12) Patent Application Publication
(10) Pub. No.: US 2003/0144558 A1
(43) Pub. Date: Jul. 31, 2003

(54) Method for producing optionally substituted aliphatic, aromatic or heteroaromatic aldehydes

(76) Inventors: Wolfram Hendel, Leonding (AT); Ferdinand Heu, Linz (AT); Peter Poechlauer, Linz (AT); Berthold WInkler, Steyr (AT)

Correspondence Address: WENDEROTH, LIND & PONACK, L.L.P., 2033 K STREET N. W., SUITE 800, WASHINGTON, DC, 20006-1021, US

(21) Appl. No.: 10/311,272

(22) PCT Filed: Jun. 8, 2001
(86) PCT No.: PCT/EP01/06530

(30) Foreign Application Priority Data
Jul. 5, 2000 (AT) A 1152/2000

(51) Int. Cl. C07D 213/46; C07C 45/00

(52) U.S. Cl. 568/435; 546/315; 568/465; 548/530

(57) ABSTRACT

The invention relates to a method for producing optionally substituted aliphatic, aromatic or heteraromatic aldehydes of formula (I), whereby the R represents a C₁-C₂₀ Alkyl radical, an aromatic or heteraromatic radical Ar which can optionally be substituted once or on a number of occasions by OH, C₁-C₆ alkyl, C₁-C₆ alkoxy, C₁-C₆ carboxylic acids or ester containing 1-6 C atoms in the ester part, phenyl, halogen, SO₃H, NO₂, NR₁R₂ or SR₁ whereby R₁ and R₂ can be independently H, phenyl or C₁-C₆ alkyl. The invention is characterised by a compound of formula (II) wherein R has the above meaning, a) is diazotized in an acidic medium, at a temperature of between −10 −+100 ° C. by a diazoation reagent and is transformed into the corresponding hydroxy carboxy acid whereby b) is transformed, by means of oxygen, into the appropriate aldehyde of formula (I) in the presence of a metal, the salt thereof, oxide or hydroxide as a catalyst.

Aliphatic and aromatic aldehydes represent important intermediates in the chemical, pharmaceutical and cosmetics industry, with the result that a number of methods for producing same are already known from the literature, although these are unsatisfactory for industrial exploitation.

These are, in addition to the variants of the Reimer-Tiemann reaction (Chem. Rev. 60 (1960), 169), for example under pressure, with transition catalysts or with cyclodextrins, the hydroxymethylation of phenol with subsequent oxidation, and also the reaction of phenol with glyoxylic acid and subsequent oxidative decarboxylation in the presence of metal salts of the mandelic acid derivatives obtained as intermediates (e.g. U.S. Pat. No. 2,640,083).

Chem. Abstr. (1982): 597981 discloses the oxidation of hydroxyphenylglycine to hydroxybenzaldehyde using metal catalysts. However, as comparative experiments have shown, reaction control is very difficult due to the high proportion of by-products.

Surprisingly, it has now been found that optionally substituted aliphatic, aromatic and heteroaromatic amino acids can be reacted by means of a one-pot synthesis by diazotization and subsequent oxidative decarboxylation to give the corresponding aldehydes in high yield and purity.

Accordingly, the invention provides a method for producing optionally substituted aliphatic, aromatic and heteroaromatic aldehydes of the formula

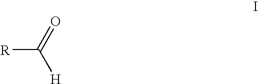

in which R is a C₁-C₂₀-alkyl radical, an aromatic or heteroaromatic radical Ar which may optionally be mono- or polysubstituted by OH, C₁-C₆-alkyl, C₁-C₆-alkoxy, C₁-C₆-carboxylic acids or esters having 1-6 carbon atoms in the ester moiety, phenyl, halogen, SO₃H, NO₂, NR₁R₂ or SR₁, where R₁ and R₂ may, independently of one another, be H, phenyl or C₁-C₆-alkyl, which is characterized in that a compound of the formula

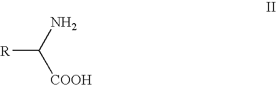

in which R has the above meaning,

a) is diazotized in an acidic medium using a diazotization reagent at a temperature of from −10 to +100° C., and converted into the corresponding hydroxycarboxylic acid, after which

b) the latter is reacted with oxygen in the presence of a metal, its salt, oxide or hydroxide as catalyst to give the corresponding aldehyde of the formula I.

In the method according to the invention, compounds of the formula II are converted to the corresponding aldehydes of the formula I.

Suitable compounds of the formula II here are a-amino acids which have an optionally substituted aliphatic, aromatic or heteroaromatic radical.

Aliphatic radicals here are C₁-C₂₀-alkyl radicals which may be linear, branched or cyclic, such as, for example, methyl, ethyl, n-propyl, isopropyl, n-butyl, sec-butyl, tert-butyl, pentyl, hexyl, octyl, decyl, dodecyl, cyclopentyl, cyclohexyl, cyclooctyl, etc. Preference is given to C₁-Cl₂-alkyl radicals, particular preference to C₁-C₆-alkyl radicals.

Aromatic and heteroaromatic radicals Ar here are radicals derived from aromatics or heteroaromatics having one or more heteroatoms or from condensed ring systems which may have one or more heteroatoms, such as, for example, benzene, pyrrole, furan, thiophene, pyridine, pyran, thiopyran, pyrimidine, pyridazine, indene, imidazole, pyrazole, thiazole, oxazole, naphthalene, anthracene, quinoline, isoquinoline, benzo(g)isoquinoline, indole, coumarone, thionaphthene, acridine etc.

Preferably, Ar is an aromatic radical or a condensed ring system with at most one heteroatom, such as, for example, phenyl, pyrrolyl, pyridinyl, thiophenyl, naphthyl, etc. Particular preference is given to aromatic radicals with only one ring and at most one heteroatom phenyl, pyrrolyl, pyridinyl, thiophenyl etc.

The aliphatic, aromatic and heteroaromatic radicals are here optionally substituted by one or more substituents from the group consisting of OH, linear, branched or cyclic C₁-C₆-alkyl or C₁-C₆-alkoxy radicals, C₁-C₆-carboxylic acids or esters having 1-6 carbon atoms in the ester moiety, phenyl, halogen, SO₃H, NO₂, NR₁R₂ or SR₁, where R₁ and R₂, independently of one another, may be H, phenyl or a linear, branched or cyclic C₁-C₆-alkyl radical.

Preferred substituents are OH, C₁-C₄-alkoxy, such as methoxy, ethoxy, propoxy and butoxy, halogens, such as F, Cl, Br and I, and NR₁R₂ or SR, where R₁ and R₂ are C₁-C₄-alkyl. Particular preference is given to OH and C₁-C₂-alkoxy.

For the method according to the invention, very particular preference is given to using hydroxyphenylglycines or alkoxyphenylglycines, such as p-hydroxyphenylglycine.

The starting compounds can be used here as racemate or in enantiomerically pure form as R or S enantiomer.

The conversion of the starting compounds of the formula II to the corresponding aldehydes of the formula I takes place in a one-pot synthesis in two steps.

In step a), a compound of the formula II is reacted in an acidic medium to give the corresponding x-hydroxycarboxylic acid.

The diazotization agents used are customary diazotization agents, such as, for example, NaNO₂ or isopentyl nitrite. Preference is given to using NaNO₂ as diazotization agent. The diazotization agent is used here in an equimolar amount or in a molar excess. Preference is given to using a 1 to 50% molar excess, particularly preferably a 5 to 30% molar excess, of diazotization agent, based on the compound of the formula II used.

Step a) takes place in an acidic medium. To prepare the acidic medium, water in combination with an inorganic acid is suitable. Preferred inorganic acids here are hydrochloric acid and sulfuric acid.

For the diazotization step, a pH of <6, preferably <2, is set in accordance with the invention.

The reaction temperature is between −10 and +100° C., preferably 0 to 80° C. and particularly preferably +10 to 70° C. The temperature profile during step a) may proceed here, for example, in 2 stages, such that firstly, for the diazotization, a lower temperature, for example −10 to +70° C., preferably up to +60° C., is established, and then for conversion to the corresponding hydroxycarboxylic acid (boiling down) the temperature is increased, for example to 40 to 100° C., preferably up to 80° C. The diazotization and the conversion (i.e. the boiling down) to give the corresponding hydroxycarboxylic acid may, however, also be carried out simultaneously if step a) is carried out at a temperature between about 40 and 80° C., preferably at about 50 to 60° C.

The corresponding α-hydroxycarboxylic acid which is formed in step a) remains in the reaction mixture and is converted to the desired aldehyde of the formula I without prior isolation in step b) by oxidative decarboxylation by means of oxygen in the presence of metals, salts thereof (e.g.: chlorides, sulfates, nitrates), oxides or hydroxides as catalysts.

In addition to the conversion to the corresponding hydroxycarboxylic acid, an oxidative decarboxylation to give the corresponding aldehyde of the formula (I) also sometimes even arises during the diazotization and the boiling down depending on the reaction parameters chosen. This produces a mixture of hydroxycarboxylic acid and aldehyde or directly the desired aldehyde, meaning that step b) may be dispensed with.

If step b) is carried out after step a), then the oxidative decarboxylation takes place by means of oxygen. Oxygen can be used here in the form of, pure oxygen, in the form of air or in the form of an N₂/O₂ mixture.

Suitable catalysts are customary metals known from the prior art, for example from U.S. Pat. No. 2,640,083, from Chem. Abstr. (1982): 597981 or from DE 2930222, or their salts, such as chlorides, sulfates, nitrates, phosphates, their oxides or hydroxides. Suitable metals are, for example, copper, iron, cobalt, manganese, chromium, lead, cerium, iridium, nickel, mercury, bismuth, zinc, aluminum, vanadium, selenium, tellurium, tungsten and antimony. Preferred metals are copper, iron, cobalt, manganese. Suitable salts are, for example, copper(I) chloride, copper(II) chloride, copper(II) sulfate, iron(II) chloride, iron(III) chloride, iron(II) sulfate, iron(III) sulfate, iron(III) nitrate, cobalt(III) sulfate, cobalt(III) nitrate, manganese(II) sulfate, manganese(II) chloride, manganese(II) acetate, etc. The metals are preferably used in the form of an inorganic and/or organic salt, particularly preferably in the form of the chloride or sulfate.

Particularly preferred metals here are copper or iron. The catalyst is preferably used in a concentration of 1 mol % to equimolar. Particular preference is given to concentrations of 5-50 mol %.

The oxidative decarboxylation can be carried out in the same medium as in step a). It is, however, also possible to render the pH of the reaction mixture basic by adding a suitable base, such as, for example, NaOH, KOH, CaO or Ca(OH)₂.

Preferably, for step b), the pH of the reaction solution is adjusted to >7, particularly preferably to a value between 9 and 14.

The oxidative decarboxylation can be carried out at atmospheric pressure or at elevated pressure. Accordingly, preference is given to establishing a pressure between 1 and 7 bar, particularly preferably between 1 and 5 bar.

The reaction temperatures in step b) are, depending on the pressure chosen, between 5 and 200° C., preferably between 15 and 150° C. and particularly preferably between 50 and 140° C.

The desired aldehyde of the formula I is isolated, depending on the aggregate state, by customary isolation methods, such as, for example, extraction, distillation, or crystallization.

To increase the yield of aldehyde, unreacted hydroxycarboxylic acid can be separated from the aldehyde, for example by basic extraction, and then optionally subjected again to step b).

The method is preferably suitable for producing aldehydes of the formula I in which the radical R is an aromatic five- or six-membered ring with at most one heteroatom or a condensed ring system with at most one heteroatom, each of which is substituted by OH, C₁-C₄-alkoxy, such as methoxy, ethoxy, propoxy and butoxy, halogens, such as Fl, Cl, Br, I, or NR₁R₂ and SR, where R₁ and R₂ are H or C₁-C₄-alkyl, particularly preferably by OH and C₁-C₂-alkoxy.

The method according to the invention is very particularly preferably used to prepare hydroxybenzaldehydes or alkoxybenzaldehydes, such as p-hydroxybenzaldehyde or p-methoxybenzaldehyde.

The aldehydes of the formula I are obtained here in a simple one-pot synthesis in high yields and with high purity.

### EXAMPLE 1

20 ml of an aqueous solution of sodium nitrite (2.2 M) were added in portions, at 50° C., to a solution of 4-hydroxyphenylglycine (6.69 g; 40 mmol) in 100 ml of semiconcentrated hydrochloric acid, and the mixture was stirred for one hour. For complete conversion, a further 4.3 mmol of NaNO₂ in 5 ml of water were added. The solution was extracted with MTBE (3×100 ml) and the 4-hydroxymandelic acid which remained in the organic phase was separated from 4-hydroxybenzaldehyde (remains in the organic phase) by extraction at pH 6-7. 4-Hydroxymandelic acid was obtained by repeated extraction of the acidified aqueous solution (pH<3). At 99% conversion, 60 mol % of 4-hydroxymandelic acid and 25 mol % of 4-hydroxybenzaldehyde with a purity of 95% were obtained.

### EXAMPLE 2

In a 100 ml three-necked round-bottomed flask (high-efficiency condenser, oxygen inlet pipe), 4-hydroxymandelic acid (4.16 g; 20 mmol, from Example 1) and copper(II) chloride (2.69 g; 20 mmol) were dissolved in water (60 ml), treated with aqueous NaOH (40% strength; 2 ml) (pH=9-9.5) and heated to 90° C. With vigorous stirring, oxygen (40 ml/min) was introduced into the solution. After 13 hours, the reaction solution was adjusted to pH 1 with aqueous HCl and the solution was extracted with CHCl₃ (4×100 ml). The organic phases were combined, dried over NaSO₄ and concentrated by rotary evaporation. 4-Hydroxybenzaldehyde was obtained as a slightly beige powder (2.28 g, 93%) with a purity of 89.4% by weight.

### EXAMPLE 3

At about 0° C., 30 ml of an aqueous solution of sodium nitrite (66 mmol) were added in portions to a solution of 4-hydroxyphenylglycine (10.03 g; 60 mmol) in 170 ml of semiconc. hydrochloric acid, and the mixture was stirred for one and a half hours. Following complete conversion, 69.4% of hydroxymandelic acid and 8.3% of 4-hydroxybenzaldehyde were found in the reaction solution. Copper(II) chloride (0.67 g, 5 mmol) was added to this solution and then oxygen (25-30 ml/min) was bubbled in at 70-80° C. After 14 hours, the mixture was extracted with MTBE and incompletely reacted hydroxymandelic acid was removed by washing the organic phase at pH 7. 5.1 g of 4-hydroxybenzaldehyde with a purity of 90.1% by weight (yield: 63%) were obtained from the organic phase which remained.

1. A method for producing optionally substituted aliphatic, aromatic and heteroaromatic aldehydes of the formula 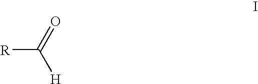
in which R is a C₁-C₂₀-alkyl radical, an aromatic or heteroaromatic radical Ar which may optionally be mono- or polysubstituted by OH, C₁-C₆-alkyl, C₁-C₆-alkoxy, C₁-C₆-carboxylic acids or esters having 1-6 carbon atoms in the ester moiety, phenyl, halogen, SO₃H, NO₂, NR₁R₂ or SR₁, where R₁ and R₂ may, independently of one another, be H, phenyl or C₁-C₆-alkyl, characterized in that a compound of the formula 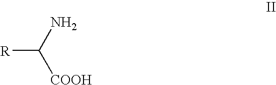
in which r has the above meaning,
a) is diazotized in an acidic medium using a diazotization reagent at a temperature of from −10 to +100° C., and converted into the corresponding hydroxycarboxylic acid, after which
b) the latter is reacted with oxygen in the presence of a metal, its salt, oxide or hydroxide as catalyst to give the corresponding aldehyde of the formula I.

2. The method as claimed in claim 1, characterized in that step a) is carried out at a pH of <6 and step b) is carried out at a pH of >7.

3. The method as claimed in claim 1, characterized in that, in step a), the diazotization takes place at a temperature of from −10 to +70° C., and the temperature is then increased to from 40 to 100° C. for conversion to the corresponding hydroxycarboxylic acid.

4. The method as claimed in claim 1, characterized in that the diazotization and the conversion into the hydroxycarboxylic acid takes place simultaneously at a temperature between 40 and 80° C.

5. The method as claimed in claim 1 characterized in that the oxidative decarboxylation is carried out at a pressure between 1 and 7 bar and at a temperature between 5 and 200° C.

6. The method as claimed in claim 1, characterized in that the oxidative decarboxylation takes place as early as in step a), meaning that in step a) either a mixture of aldehyde of the formula (I) and the corresponding hydroxycarboxylic acid, or directly the aldehyde of the formula (I) is obtained.

7. The method as claimed in any of claims 1 to 6, characterized in that, to increase the yield of aldehyde of the formula (I), unreacted hydroxycarboxylic acid is separated off from the aldehyde by basic extraction and optionally subjected to step b) again.

8. The method as claimed in claim 1, characterized in that aldehydes of the formula (I) in [lacuna] R is a C₁-C₁₂-alkyl radical or an aromatic radical having at most one heteroatom or a condensed ring system with at most one heteroatom, which may optionally be mono- or polysubstituted by OH, C₁-C₄-alkoxy, halogen, NR₁R₂ or SR₁ where R₁ and R₂ are H or C₁-C₄-alkyl, are prepared.

9. The method as claimed in claim 1, characterized in that hydroxy- or alkoxybenzaldehydes are obtained.
